(19) United States
(12) Patent Application Publication
(10) Pub. No.: US 2010/0273822 A1
(43) Pub. Date: Oct. 28, 2010

(54) Immediate release compositions and methods for delivering drug formulations using strong acid ion exchange resins

(76) Inventors: William Wayne Howard, Morristown, NJ (US); Russell Francis Somma, Sparta, NJ (US)

Correspondence Address: SHELDON KAVESH, Ph. D., 16 N. POND RD., WHIPPANY, NJ, 07981, US

(21) Appl. No.: 12/799,259

(22) Filed: Apr. 21, 2010

Related U.S. Application Data
(60) Provisional application No. 61/171,612, filed on Apr. 22, 2009.

(51) Int. Cl. A61K 31/485; A61K 31/137; A61K 47/48; A61P 31/04; A61P 1/00

(52) U.S. Cl. 514/282; 514/653

(57) ABSTRACT

Solid oral dosage immediate release compositions comprising strong acid ion exchange resins and methods for delivering drug formulations.

### CROSS REFERENCE TO RELATED APPLICATIONS

This application claims the benefit of U.S. Provisional Application Ser. No. 61171612 filed Apr. 22, 2009.

### BACKGROUND OF THE INVENTION

The present invention relates to the use of strong acid ion exchange resins (IER) to create an immediate release (IR) drug delivery system using release enhancers.

Formulations containing weak acid ion exchange resins are frequently used for immediate release of pharmaceutical agents in a patient's stomach. However, release from weak acid resins is slowed and reduced at higher than normal stomach pH levels. High pH levels could occur if the patient is taking medications such as proton pump inhibitors (PPIs) or has a disease state that induces hypochlorhydria or achlorhydria. In either case, a weak acid formulation may not release the medicament at a rate or to an extent adequate to achieve the desired therapeutic effect.

Approximately 60 million prescriptions were written for PPIs in 2006. Additionally, in the U.S., another 10 million people were reported to have self medicated with PPIs in 2008. Furthermore, about one in three adults used antacids on a regular basis. Collectively, these statistics suggest that close to 100 million people in the U.S. could be taking a drug that could significantly interfere with the release profile of a weak acid IER formulation.

The history of prior art dosage forms indicates that a serious need exists for a novel and useful solid oral dosage form that provides an unexpected advancement in the science of IER dosage forms. For example, prior art dosage forms lack the ability to provide the immediate release properties of weak acid IER formulations while using a strong acid resin. Surprisingly and unexpectedly, useful strong acid resinates can be formulated to have immediate release characteristics. The present invention creates a release enhancing strong acid resin drug formulation by adding a release enhancing agent to the formulation to increase the rate and extent of drug release from the formulation such that it meets an a priori definition of immediate release.

### SUMMARY OF THE INVENTION

In one embodiment, the invention is a solid oral dosage pharmaceutical composition comprising (i) at least one pharmaceutically active agent bound to a first strong acid ion exchange resin to form a strong acid ion exchange resinate and (ii) a release-enhancing agent; wherein said composition is capable of immediate release of said at least one pharmaceutically active agent from said strong acid ion exchange resinate.

Immediate release when administered to a patent is defined as at least 80% release of a pharmaceutically active agent within 45 minutes in a standard dissolution apparatus according to the USP 31 NF 26 section 711.

In another embodiment, the invention is a method of orally administering to a patient a solid dosage pharmaceutical composition comprising (i) at least one pharmaceutically active agent bound to a strong acid ion exchange resin to form a strong acid ion exchange resinate and (ii) a release enhancing agent, wherein said administration results in immediate release of said pharmaceutically active agent from said strong acid ion exchange resinate.

By adding a release enhancing agent with a strong affinity for the ionic resin to the strong acid resin drug formulation, much more rapid release of a resinated drug can be attained in normal gastric fluid than otherwise would occur without the presence of the release enhancing agent. Further, the added release enhancing agent with a strong acid ion exchange resin formulation can also facilitate release of the resinated drug in abnormal human gastric fluid wherein the pH is much higher than normal due to the use of drugs such as PPI or the presence of disease states such as H. pylori or atrophic gastritis that can lead to hypochlorhydria and achlorhydria.

Thus, one can attain the rapid release properties of weak acid resinates with a strong acid resinate while retaining the low sensitivity to pH change associated strong acid resins and the high drug loading of strong acid resins by adding a release enhancing agent to the strong acid drug formulation.

The addition of a release enhancing agent to the strong acid IER formulation also avoids using very small particles to speed up drug release. If a strong acid resin is used in conjunction with a release enhancing agent, larger particles can be used and still obtain rapid drug release thereby avoiding the need to use smaller particles for faster drug release. Therefore, manufacturing will be simpler since there will be no need for a size classification step and subsequent disposal of unwanted size fractions during the manufacturing operation and issues of unwanted particle agglomeration will be avoided.

It is an object of the present invention to create a strong acid resin formulation that retains the benefits of such strong acid formulations while providing immediate release of the drug not normally associated with strong acid resin formulations and simultaneously over coming the low loading and pH related drug release properties of weak acid resin drug formulations.

### BRIEF DESCRIPTION OF THE DRAWINGS

FIG. 1 contains a comparison of the dissolution of two pharmaceutical compositions, one with 40 mg of CaCl₂, which is a release enhancing agent, and a second formulation without a release enhancing agent.

FIG. 2 contains a comparison of the dissolution of two pharmaceutical compositions, one with 60 mg of CaCl₂, CaCl₂ is a release enhancing agent, and a second formulation without a release enhancing agent.

FIG. 3 is a flow chart that illustrates a process for creating a drug/resin complex, the drug resination process.

FIG. 4 is a flow chart that illustrates a process for adding a coating to a drug resinate to enhance material flow during additional manufacturing processes.

FIG. 5 is a flow chart that illustrates a process for adding an extended release coating to a drug resinate.

FIG. 6 is a flow chart that illustrates a process for creating a second drug component which may be added to a drug resinate formulation.

FIG. 7 is a flow chart that illustrates a process for creating a release enhancer which may be added to a drug resinate formulation.

FIG. 8 is a flow chart that illustrates a process for creating an immediate release capsule dosage form containing a drug resinate and a release enhancing agent.

FIG. 9 is a flow chart that illustrates a process for creating an immediate release tablet dosage form containing a drug resinate and a release enhancing agent.

FIG. 10 is a flow chart that illustrates a process for creating an immediate release tablet dosage form containing a drug resinate, a second drug component and a release enhancing agent.

FIG. 11 is a flow chart that illustrates a process for creating an immediate release capsule dosage form containing an immediate release drug resinate, a second drug component and a release enhancing agent.

FIG. 12 is a flow chart that illustrates a process for creating a capsule dosage form containing an extended release drug resinate, an immediate release resinate component and a release enhancing agent.

FIG. 13 is a flow chart that illustrates a process for creating an extended release pseudoephedrine component by coating immediate release pseudoephedrine particles.

FIG. 14 is a flow chart that illustrates a process for creating a fixed combination ER tablet by combining IR and ER resinates along with IR and ER pseudoephedrine.

FIG. 15 is a flow chart that illustrates a process for creating a fixed combination ER capsule by combining IR and ER resinates along with IR and ER pseudoephedrine.

### DETAILED DESCRIPTION OF THE INVENTION

The invention comprises a solid oral dosage form, such as a tablet, caplet, capsule, orally disintegrating tablet, powder, consumable film or any other dosage form, containing at least one pharmaceutical agent bound to an ion exchange resin and at least one release enhancing agent, and methods for using this dosage form. The composition is designed to be swallowed without forming a liquid state of the active drug substance prior to ingestion through the oral cavity and into the gastric system.

In one embodiment, the invention is a solid oral dosage pharmaceutical composition comprising (i) at least one pharmaceutically active agent bound to a first strong acid ion exchange resin to form a strong acid ion exchange resinate and (ii) a release-enhancing agent; wherein said composition is capable of immediate release of said at least one pharmaceutically active agent from said strong acid ion exchange resinate.

The strong acid ion exchange resin may be, for example, Amberlite IRP69, DOWEX 88, or DOWEX 50WX8). The release enhancing agent can be, for example, an inorganic salt (e.g., a salt including Fe³⁺, Ca²⁺, Mg²⁺, or Fe²⁺) an organic base (e.g., thiamine, guanine, or cytosine), a cationic surfactant (e.g., cetyltrimethylammonium bromide (CTAB), denatonium benzoate, or benzalkonium chloride), or a non-ionic surfactant (e.g., Tween 20 or Tween 80).

When administered to a patient, the release enhancing agent results in immediate release of the pharmaceutically active agent(s) from the strong acid ion exchange resinate. The pharmaceutical composition can be formulated, for example, as a capsule or compressed tablet.

The pharmaceutical composition can optionally include a second ion exchange resin. This second ion exchange resin is bound to one or more pharmaceutical agents and can be coated with a extended release coating, resulting in extended release of the pharmaceutically active agent(s) from the second ion exchange resinate when administered to a patient. The second ion exchange resin can be bound to the same or different pharmaceutically active agent as the first ion exchange resin.

In another embodiment, the invention is a method of orally administering to a patient a solid dosage pharmaceutical composition comprising (i) at least one pharmaceutically active agent bound to a strong acid ion exchange resin to form a strong acid ion exchange resinate and (ii) a release enhancing agent, wherein said administration results in immediate release of said pharmaceutically active agent from said strong acid ion exchange resinate.

In one aspect, the invention comprises a method of orally administering one of the foregoing pharmaceutical compositions to a patient (e.g., a patient with a Helicobacter pylori infection or atrophic gastritis), resulting in immediate release of the pharmaceutically active agent from the strong acid ion exchange resinate.

In another aspect, the invention comprises a method of delivering a pharmaceutically active agent to a patient, the method comprising orally administering one of the foregoing pharmaceutical compositions and a compound selected from the group consisting of a proton pump inhibitor, an H2 receptor antagonist, and an antacid, wherein said administration results in immediate release of the pharmaceutically active agent from the strong acid ion exchange resinate; and wherein the pharmaceutical compositions is administered within 24 hours of said proton pump inhibitor, H2 receptor antagonist, or antacid.

In yet another aspect, the invention comprises a method of delivering any of the foregoing pharmaceutical compositions to a patient with hypochlorhydria or achlorhydria in the stomach (e.g., hypochlorhydria or achlorhydria resulting from disease or the use of a proton pump inhibitor, H2 receptor antagonist, or antacid).

By “release enhancing agent” is meant an agent that, when added to a strong acid drug resin formulation, increases the rate and extent of drug release than would otherwise occur without the release enhancing agent in the same formulation.

By “pharmaceutically active agent” is meant articles intended for use in the diagnosis, cure, mitigation, treatment, or prevention of disease in man or other animals and articles (other than food) intended to affect the structure or any function of the body of man or other animals that are physiologically acceptable. The agent could be a combination of pharmaceutical active agents as well as a single agent.

By “pharmaceutical composition” is meant the pharmaceutically active agent(s), the release enhancing agent(s) and other pharmaceutical materials known to those skilled in the art needed to create a solid oral dosage form.

By “physiologically acceptable” is meant those substances that are adequately tolerated without causing unacceptable negative side effects.

By “ion exchange resin” is meant an insoluble solid matrix that carries exchangeable ions with either a positive or negative charge. The trapping of ions takes place only with simultaneous releasing of other ions. Ions are exchanged in stoichiometrically equivalent amounts of other ions with the same electrical charge when the ion exchange material is in contact with an electrolytic solution.

By “resinate” or “drug resinate” is meant the complex formed when a drug exchanges an ion with a resin particle in the stoichiometric process described above and a drug/resin compound is formed.

By “strong acid ion exchange resin” is meant a cation exchange resin in which the ion exchange groups are completely dissociated at all pHs. As an example, in a strong acid resin the ionizable group introduced to the polymer is a sulfonic acid group (SO₃H) as opposed to the carboxylic acid (COOH) used in weak acid resins.

By “weak acid ion exchange resin” is meant a cation exchange resin in which the ion exchange groups are incompletely dissociated at some pHs. As an example, in a weak acid resin the ionizable group introduced to the polymer is a carboxylic acid (COOH) as opposed to the sulfonic acid group (SO₃H) used in strong acid resins.

By “immediate release” is meant that the pharmacologically active agent is released from the formulation immediately such that 80%, 85%, 90%, or even 95% of the pharmaceutically active agent in the formulation is released within 45 minutes when dissolution is measured according to the USP 31 NF 26 section 711.

By “extended release” is meant that the pharmaceutically active agent is released from the formulation at a controlled rate such that the formulation allows for a reduction in dosing frequency as compared to that presented by a conventional dosage form, e.g. an immediate release dosage form.

The present invention comprises a pharmaceutical composition including a strong acid ion exchange resin (LER) to achieve an immediate release (IR) of one or more pharmaceutically active agents. The pharmaceutical compositions of the invention comprise the combination of a strong acid resin with a release enhancing agent to achieve faster, and more complete, drug release compared to a strong acid resin formulation without the release enhancing agent.

The invention comprises both compositions including a single pharmaceutically active agent and fixed combination products containing a second pharmaceutically active agent. Furthermore, the invention comprises compositions including both an IR component and extended release (ER) component. In these embodiments, the IR component (as described above) can also be used as the nucleus to provide the extended release component through the use of an extended release coating.

Whether for extended release applications or immediate release applications, strong acid resins have several superior properties for IER drug formulations as compared to weak acid resins. For example, strong acid resin release kinetics are virtually unaffected by change in the pH of the dissolution environment as compared to weak acid resins. The drug release kinetics of weak acid resins can be affected by higher pH levels in the gastric fluid such that the rate and extent of drug release can be greatly reduced.

Further, a strong acid resin dosage form is useful for assuring that the resinated drug is released from an MR formulation when stomach acid is reduced or eliminated (hypochlorhydria and achlorhydria) by disease states such as Helicobacter (H.) pylori infection or atrophic gastritis.

Furthermore, a strong acid resin dosage form provides high drug loadings which lead to (1) less wasted active pharmaceutical ingredient (API), (2) lower disposal costs for controlled substances, (3) easier compliance with DEA and environmental regulations, and (4) smaller oral solid dosage forms due to less bulk of the material. Smaller oral solid dosage forms are easier for the patient to swallow and may improve patient compliance.

Formulations containing strong acid resin immediate release dosage forms can use larger particles, thereby eliminating sizing during manufacturing and disposal of unwanted size fractions. Undesirable particle agglomeration is also reduced or avoided while still retaining immediate release kinetics.

By adding a release enhancing agent with a strong affinity for the ionic resin to the strong acid resin drug formulation, more rapid release of a resinated drug can be attained in normal gastric fluid than otherwise would occur without the presence of the release enhancing agent. Further, the added release enhancing agent with a strong acid resin formulation can also facilitate release of the resinated drug in abnormal human gastric fluid where the pH is much higher than normal due to the use of drugs such as proton pump inhibitors or the presence of disease states such as H. pylori infection or atrophic gastritis.

Release Enhancing Agents

The drug-containing strong acid ion exchange resins of the invention are formulated with release enhancing agents. These release enhancing agents result in immediate release of the drug from the strong acid ion exchange resins by interfering with drug-ion exchange bonding. Examples of suitable release enhancing agents are:

Inorganic Agents:

Fe³⁺ (e.g. FeCl₃)

Ca²⁺ (e.g. CaCl₂)

Mg²⁺ (e.g. MgCl₂)

Fe²⁺ (e.g. FeCl₂)

Organic Agents:

Thiamine

Guanine

Cytosine

Cationic Agents:

Cetyltrimethylammonium bromide (CTAB)

Benzalkonium chloride

Denatonium benzoate (Bitrex)

Non-Ionic Agents:

Polysorbate 20 (Tween 20)

Polysorbate 80 (Tween 80)

### Pharmaceutically Active Agents

The invention comprises methods and compositions for immediate release of pharmaceutically active agents using a strong acid ion exchange resin. Examples of such pharmaceutically active agents suitable for the compounds and methods of the inventions are:
A: Anti-tussives, e.g., benzonatate, caramiphen edisylate, chlophedianol, codeine, dextromethorphan hydrobromide, hydrocodone, levopropoxyphene, morphine codeine, ethylmorphine, dihydrocodeine, benzylmorphine, laudanum, dihydroisocodeine, nicocodeine, nicodicodeine, hydrocodone, hydromorphone, acetyldihydrocodeine, thebacon, diamorphine (heroin), acetylmorphone, noscapine, and pholcodine.
B: Narcotic analgesics, e.g., codeine, oxycodone, hydrocodone, diamorphine, pethidine, morphine, oxymorphone, nalorphine, naloxone, naltrexone, opium, hydromorphone, nicomorphine, dihydrocodeine, and papavereturn.
C: Decongestants, e.g., pseudoephedrine hydrochloride, phenylephrine bitartrate, and pseudoephedrine sulfate.
D: Non-steroidal anti-inflammatory drugs, e.g., aspirin, magnesium salicylate, diclofenac, etodolac, indometacin, nabumetone, sulindac, tolmetin, ibuprofen, ketoprofen, mefenamic acid, meclofenamic acid, phenylbutazone, piroxicam, meloxicam, and naproxen sodium.
E: Anti-emetic drugs, e.g., dolasetron, granisetron, ondansetron, tropisetron, palonosetron, mirtazapine, metoclopramide, cyclizine, diphenhydramine, dimenhydrinate, meclizine, promethazine, and hydroxyzine.
F: Anti-histamines, e.g., diphenhydramine, loratadine, desloratadine, meclizine, fexofenadine, pheniramine, cetirizine, promethazine, and chlorpheniramine.
G: Anti-depressants, e.g., citalopram, escitalopram, fluoxetine, fluvoxamine, paroxetine, sertraline, desvenlafaxine, duloxetine, milnacipran, venlafaxine, atomoxetine, mazindol, reboxetine, viloxazine, amitriptyline, clomipramine, doxepin, imipramine, trimipramine, desipramine, nortriptyline, protriptyline, moclobemide, phenelzine, and selegiline.
H: Tranquilizers, e.g., amobarbital, pentobarbital, secobarbital, phenobarbital, clonazepam, diazepam, estazolam, flunitrazepam, lorazepam, midazolam, nitrazepam, oxazepam, triazolam, temazepam, chlordiazepoxide, and alprazolam.
I: Anti-convulsants, e.g., felbamate, carbamazepine, oxcarbazepine, vigabatrin, progabide, tiagabine, topiramate, gabapentin, pregabalin, ethotoin, and phenyloin.
J: Hypnotics, e.g., zolpidem, zaleplon, zopiclone, and eszopiclone.
K: Muscle relaxants, e.g., methocarbamol, carisoprodol, chlorzoxazone, cyclobenzaprine, gabapentin, metaxalone, and orphenadrine.
L: Anti-psychotics, e.g., haloperidol, droperidol, chlorpromazine, fluphenazine, perphenazine, prochlorperazine, thioridazine, trifluoperazine, mesoridazine, promazine, triflupromazine, levomepromazine, methotrimeprazine, pimozide, chlorprothixene, flupenthixol, thiothixene, zuclopenthixol, clozapine, olanzapine, risperidone, quetiapine, ziprasidone, amisulpride, asenapine, and paliperidone.
M: Anti-microbials, e.g., EDTA, zinc compounds, triclosan, domiphen, cetyl pyridium chloride, domiphen bromide, fluorides, alexidine, and octenidine.
N: Anti-diarrheals, e.g., bismuth subsalicylate and loperamide.
O: CNS stimulants, e.g., caffeine, cocaine, and amphetamines.
P: Attention Deficit and Hyperactivity Disorder drugs, e.g., methylphenidate, dextroamphetamine sulfate, amphetamine, and atomoxetine hydrochloride.
The invention also includes methods and compositions for delivering combinations of pharmaceutically active compounds. Examples of such combinations are:
A: an anti-tussive and an antihistamine
B: an anti-tussive and a decongestant
C: an anti-tussive and an analgesic
D: an anti-tussive and an NSAID
E: an anti-tussive and an antihistamine and a decongestant
F: an anti-tussive and an antihistamine and an analgesic
G: an anti-tussive and an antihistamine and an NSAID
H: an anti-tussive and an antihistamine and a decongestant and an analgesic
I: a muscle relaxant and an analgesic
J: a muscle relaxant and an NSAID
K: a muscle relaxant and an analgesic and an NSAID

### L: a PPI and an NSAID

M: an H2 antagonist and an NSAID
N: a PPI and an analgesic
O: an H2 antagonist and an analgesic.

### Ion Exchange Resins

The compositions of the invention include strong acid ion exchange resins. Examples of suitable ion exchange resins are, for example,

A: Amberlite TRP69 manufactured by Rohm and Haas
B: Dowex Marathon C, Dowex 88 and Dow XYS-40010 manufactured by Dow Chemical and the like.
The examples cited above are strong acid resins and the ionizable group introduced to the polymer is a sulfonic acid group (SO₃H) as opposed to the carboxylic acid (COOH) used in weak acid resins.

### Dosage Forms

Suitable dosage forms include tablets, capsules, orally disintegrating tablets, thin films, powders, beadlets, and the like.

### EXAMPLES

### Example 1

### Formulation of a Pharmaceutical Composition

| | |
|---|---|
| Dextromethorphan Resinate* | 88 mg |
| Release enhancing agent (CaCl₂) | 40 mg |
| Magnesium Stearate | 2 mg |
| Colloidal Silicon Dioxide | 3 mg |
| HPMC 6 cps | 3 mg |
| Talc | 2 mg |
| Triethyl Citrate | 2 mg |
| Empty Capsule Shell #3 | 48 mg |
| Total Dosage Form Weight | 188 mg |

*Equivalent to Dextromethorphan Hydrobromide 30 mg

The process for creating the drug/resin complex and dosage form was as follows.

500 mg of Amberlite IRP-69 from Rohm and Haas were added to deionized water (2.5 L) which had been heated to 85° C. The resin and water were mixed using a magnetic stirring bar until a uniform suspension was obtained. 150 mg of Dextromethorphan Hydrobromide was made into a solution in deionized water and then added to the resin slurry and mixed in the primary vessel with continued mixing for 4.0 hours at 85° C. to create the dextromethorphan resinate. The slurry was vacuum filtered to separate the resinate from the water. The resin particles were washed three times by re-suspending the particles in 5 liters of deionized water maintained at 85° C. The resulting washed particles were filtered and allowed to cool for 12 hours. This process was repeated in order to generate adequate amounts of the Dextromethorphan resinate to prepare the number of capsules required for dissolution testing. Care was taken during the cooling process to avoid cake formation by periodically mixing the resinate bed with a glass stirring rod. The resinate was then dried using a lab scale fluid bed dryer set at 55° C. inlet temperature. Drying was continued until a residual moisture content of 2.0% was obtained. Drug loading was tested and showed approximately 33.9% drug load or approximately 34 mg of dextromethorphan per 100 mg of resinate.

The dried resinate was then formulated into hard gelatin capsules using the materials in the table above. The dried resinate was coated in a lab scale column coater using hydroxypropylmethylcellulose (HPMC) and triethyl citrate as a plasticizer. This is meant to enhance processing and protect the finished resin during subsequent capsule filling. The HPMC coating is a type which has no effect on the dissolution rate and is added as a processing aid to enhance material flow during additional manufacturing steps. The coated resin along with the remaining ingredients were blended in a lab scale diffusional mixer and filled into empty capsule shells.

The release enhancing agent, CaCl₂, was added to the capsule blend of the first sample while a second formulation was prepared as a control without the use of the release enhancing agent. Six capsules from each formulation were then tested for dissolution.

The test used followed USP 31 NF 26 section 711 using apparatus 1 baskets at 100 rpm (no enzymes). The media selected was simulated gastric (0.1N HCl) at a volume of 900 ml. One capsule was added to each dissolution vessel. At the 60 minute time point the baskets were removed without touching the contents and subsequently placed in dissolution vessels containing 900 ml of simulated intestinal fluid (pH of 6.8). Aliquots were drawn at 15, 30, 45, and 120 minutes. An equivalent amount of media was replaced in the dissolution vessel to account for the volume of the sample removed. The aliquots were filtered and subsequently analyzed using HPLC equipped with a UV detector. The compound is known to exhibit absorption in the range of 250-280 nM.

The following dissolution data were obtained:

### Example 1 Data

| | |
|---|---|
| IRP69 | IRP69 + CaCl₂ |

| | |
|---|---|
| Minutes | % Dextromethorphan Released |

| | | |
|---|---|---|
| 15 | 62 | 71 |
| 30 | 68 | 76 |
| 45 | 72 | 79 |
| 120 | 82 | 92 |

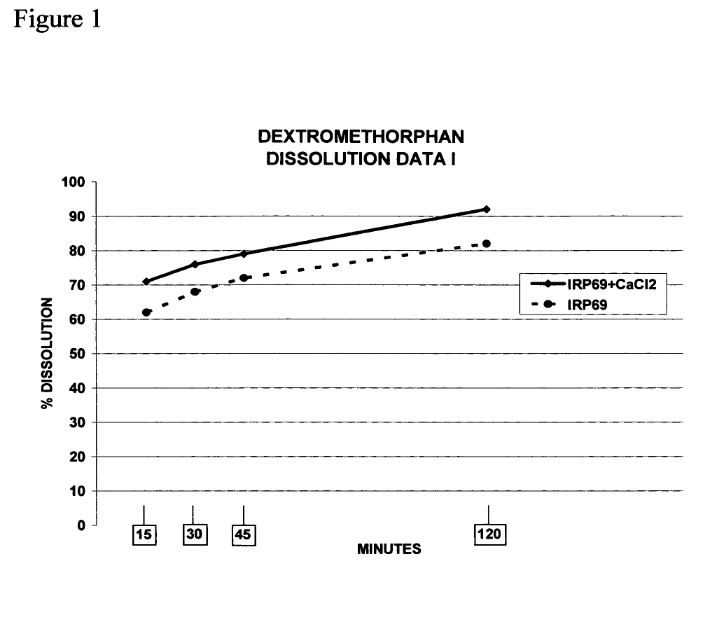

The results show that the addition of CaCl₂ increased the release rate of dextromethorphan as compared to the control formulation prepared without the addition of the release enhancing agent (REA) CaCl₂. These data are shown in FIG. 1.

### Example 2

### Formulation of a Pharmaceutical Composition

| | |
|---|---|
| Dextromethorphan Resinate* | 88 mg |
| Release enhancing agent (CaCl₂) | 60 mg |
| Magnesium Stearate | 2 mg |
| Colloidal Silicon Dioxide | 3 mg |
| HPMC 6 cps | 3 mg |
| Talc | 2 mg |
| Triethyl Citrate | 2 mg |
| Empty Capsule Shell #3 | 48 mg |
| Total Dosage Form Weight | 208 mg |

*Equivalent to Dextromethorphan Hydrobromide 30 mg.

The process that was used to create the resinate, filling the capsules and carrying out the dissolution test in this example was the same as described in Example 1 except that 60 mg of CaCl₂ was used instead of 40 mg as in Example 1. The following dissolution data were obtained:

### Example 2 Data

| | |
|---|---|
| IRP69 | IRP69 + CaCl₂ |

| | |
|---|---|
| Minutes | % Dextromethorphan Released |

| | | |
|---|---|---|
| 15 | 62 | 73 |
| 30 | 68 | 81 |
| 45 | 72 | 87 |
| 120 | 82 | 94 |

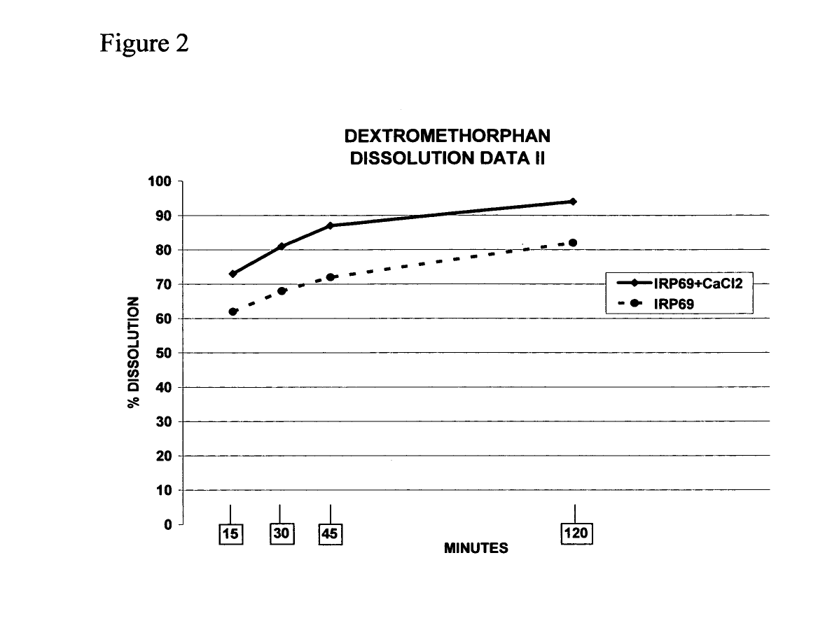

The results show that the addition of CaCl₂ increased the release rate of dextromethorphan as compared to the control formulation prepared without the addition of the release enhancing agent (REA) CaCl₂. Importantly, the results also show that release rate of the formulation with CaCl₂ meets the definition of immediate release at 45 minutes as contained herein. This strong acid resin composition was successfully modified to produce a pre-defined, immediate release rate. The release rate of dextromethorphan without the CaCl₂ does not meet the specification of immediate release. These data are shown in FIG. 2.

### Example 3

### Mono-Substance Hydrocodone IR Intermediate

| | |
|---|---|
| Hydrocodone Resinate Intermediate* | 25 mg |
| HPMC 6 cps | 3 mg |
| Talc | 2 mg |
| Triethyl Citrate | 2 mg |
| Total Dosage Form Weight | 32 mg |

*Equivalent to Hydrocodone Bitartrate 5 mg.

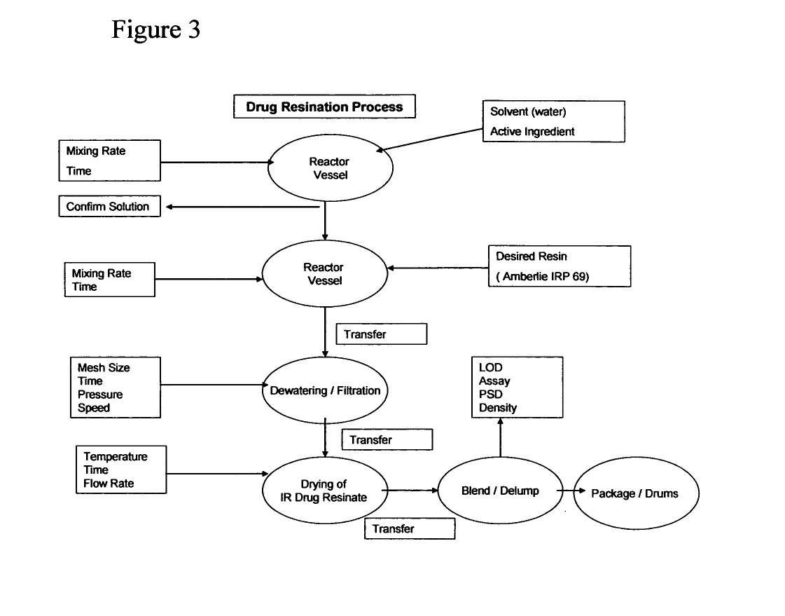

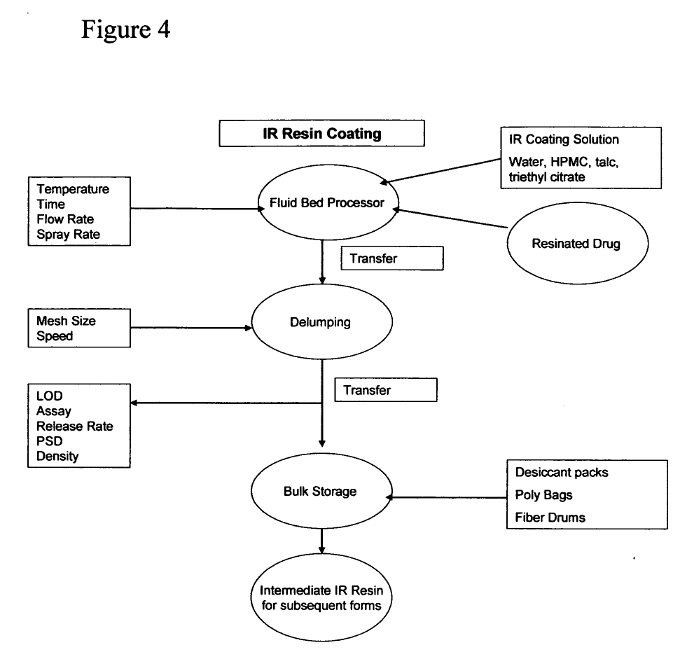

A mono-substance hydrocodone IR intermediate could be prepared as follows. The process begins with the resination of the active ingredient. The process for creating the drug resinate is illustrated in FIG. 3. Next, the hydrocodone resinate is coated to improve product flow as shown in FIG. 4.

### Example 4

### Formulation for a Commercial Hydrocodone Product: Mono-Substance IR Dosage Form, Hard Gelatin Capsule

| | |
|---|---|
| Hydrocodone Resinate Intermediate* | 32 mg |
| Release enhancing agent | 15 mg |
| Magnesium Stearate | 2 mg |
| Colloidal Silicon Dioxide | 3 mg |
| Empty Capsule Shell #3 | 48 mg |
| Total Dosage Form Weight | 100 mg |

*Equivalent to Hydrocodone Bitartrate 5 mg.

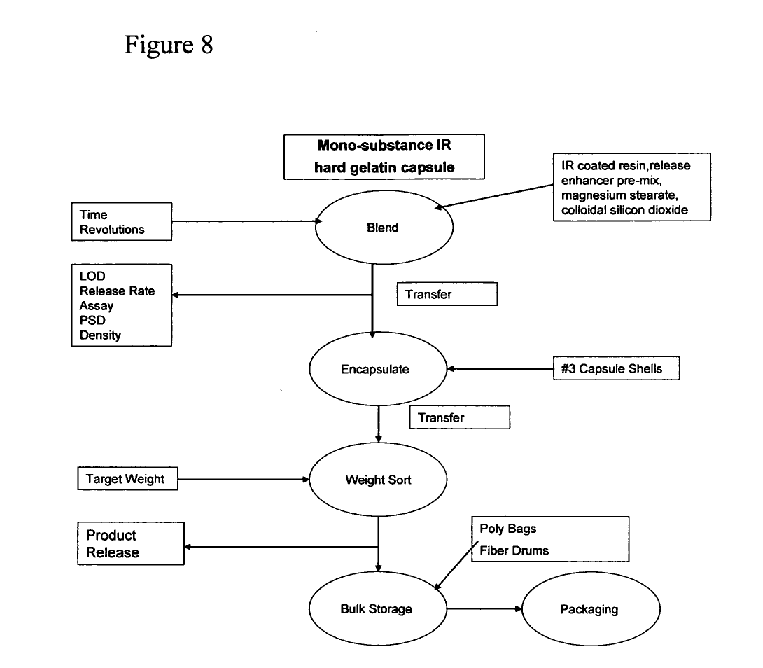

A mono-substance hydrocodone IR dosage form could be prepared as follows. The process begins with the coated hydrocodone resinate as created in Example 3 and FIG. 4. In the last step, as shown in FIG. 8, the coated resinate, the release enhancing agent and the other excipients are blended and filled into hard gelatin capsules.

### Example 5

### Formulation for a Commercial: Mono-Substance IR Hydrocodone Dosage Form, Compressed Tablet

| | |
|---|---|
| Hydrocodone Resinate Intermediate* | 32 mg |
| Microcrystalline cellulose | 150 mg |
| Polyplasdone XL | 10 mg |
| Release enhancing agent | 15 mg |
| HPMC 6 cps | 20 mg |
| Anhydrous lactose | 100 mg |
| Magnesium stearate | 4 mg |
| Total Dosage Form Weight | 331 mg |

*Equivalent to Hydrocodone Bitartrate 5 mg

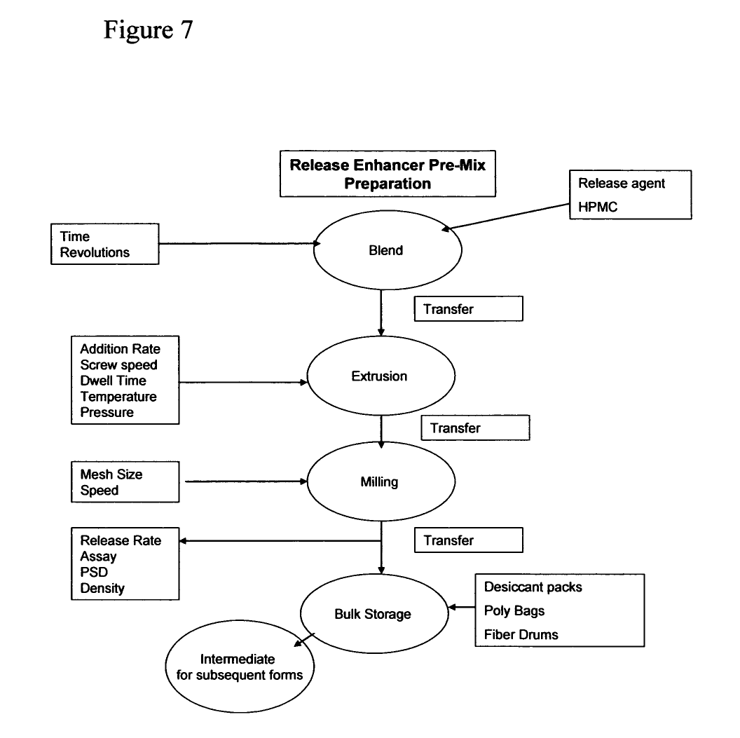

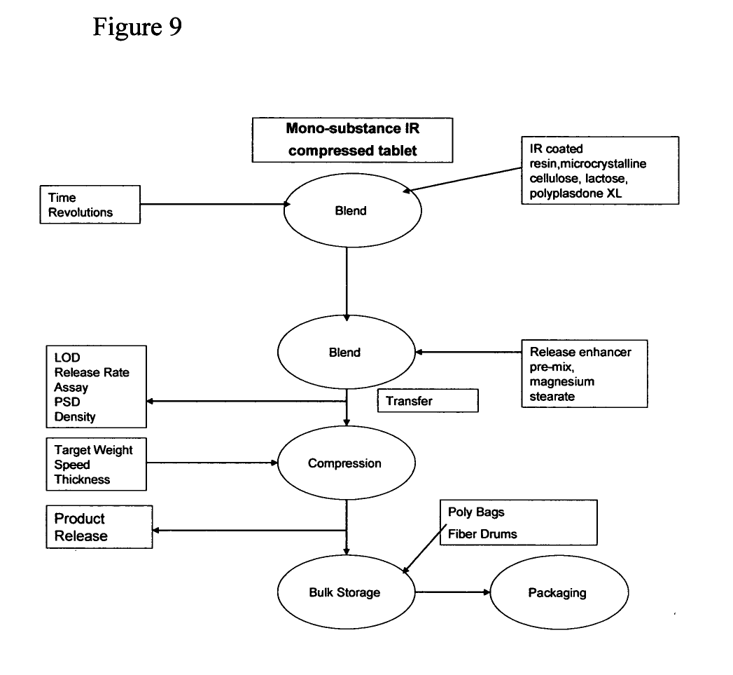

A mono-substance hydrocodone IR dosage form could be prepared as follows. The process begins with the coated hydrocodone resinate as created in Example 3 and FIG. 4. In the last step, as shown in FIG. 9, the coated resinate, the release enhancer pre-mix (prepared by a process shown in FIG. 7) and the other excipients are blended and compressed into a tablet.

### Example 6

### Mono-Substance Pseudoephedrine IR Intermediate

| | |
|---|---|
| Pseudoephedrine HCl | 60 mg |
| Microcrystalline Cellulose | 250 mg |
| Polyvinylpyrrolidone | 20 mg |
| Magnesium Stearate | 4 mg |
| Colloidal Silicon Dioxide | 4 mg |
| HPMC 6 cps | 20 mg |
| Total Dosage Form Weight | 358 mg |

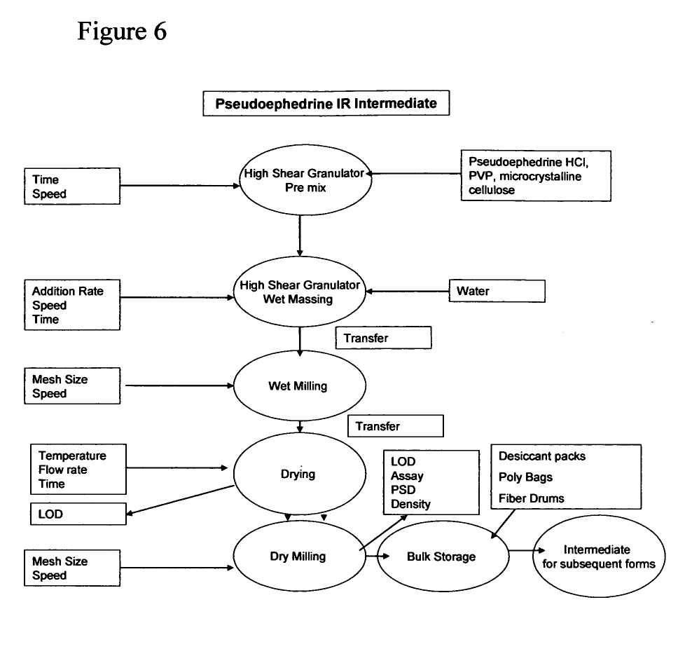

A mono-substance pseudoephedrine IR intermediate could be prepared as illustrated in FIG. 6.

### Example 7

### Formulation for a Commercial Product: Fixed Combination Dosage Form Using the IR Hydrocodone Component with a Second Medicinal Agent, Hard Gelatin Capsule

| | |
|---|---|
| Hydrocodone Resinate Intermediate* | 32 mg |
| Pseudoephedrine HCl Intermediate | 358 mg |
| Microcrystalline Cellulose | 100 mg |
| Polyvinylpyrrolidone | 20 mg |
| Release enhancing agent | 15 mg |
| Magnesium Stearate | 4 mg |
| Colloidal Silicon Dioxide | 4 mg |
| Empty Capsule Shell #1 | 76 mg |
| Total Dosage Form Weight | 609 mg |

*Equivalent to Hydrocodone Bitartrate 5 mg

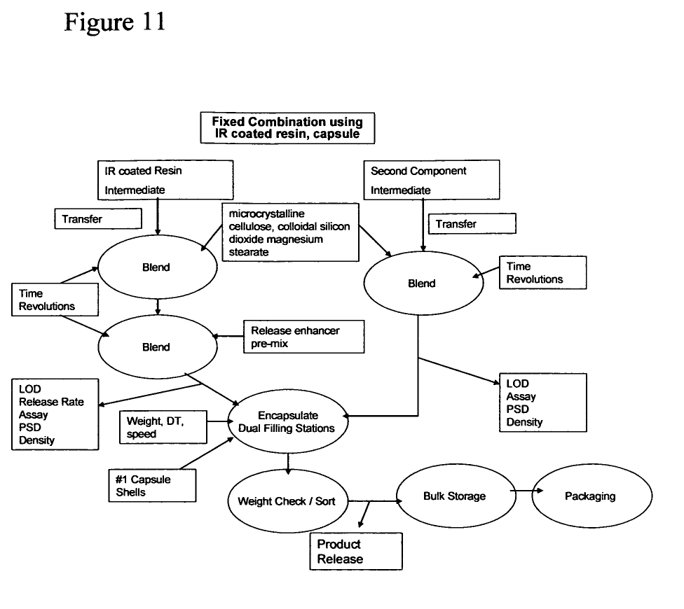

A fixed combination hydrocodone IR dosage form could be prepared as follows. The process begins with the coated hydrocodone resinate as created in Example 3 and FIG. 4. The second drug component (pseudoephedrine HCl) is created as an Intermediate as shown in Example 6 and FIG. 6. In the last step, as shown in FIG. 11, the coated resinate, the pseudoephedrine Intermediate and the release enhancing agent and the other excipients are blended and filled into hard gelatin capsules.

### Example 8

### Formulation for a Commercial Product: IR Hydrocodone Mono-Substance with a Second Medicinal Agent, Compressed Tablet

| | |
|---|---|
| Hydrocodone Resinate Intermediate* | 32 mg |
| Pseudoephedrine HCl Intermediate | 358 mg |
| Microcrystalline Cellulose | 250 mg |
| Polyvinylpyrrolidone | 20 mg |
| Release enhancing agent | 15 mg |
| Magnesium Stearate | 4 mg |
| Colloidal Silicon Dioxide | 4 mg |
| HPMC 6 cps | 20 mg |
| Total Dosage Form Weight | 703 mg |

*Equivalent to Hydrocodone Bitartrate 5 mg

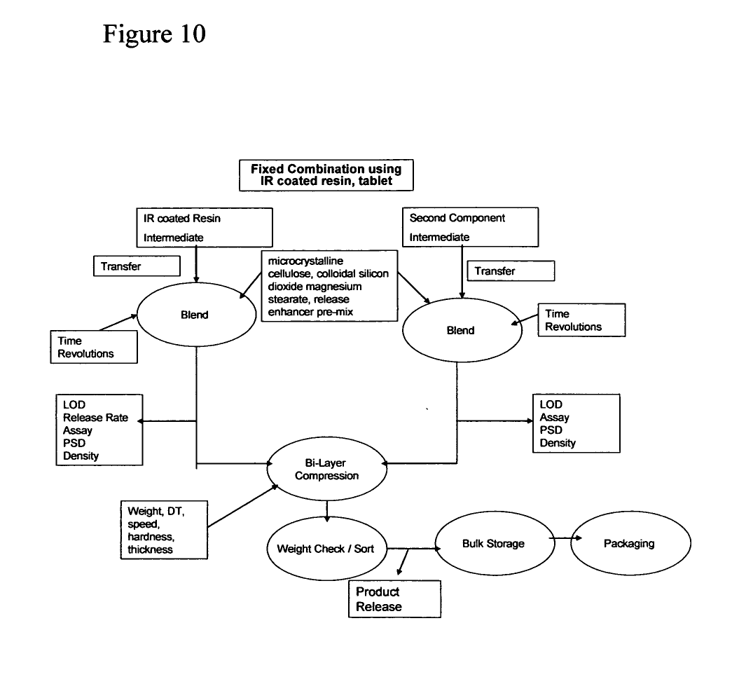

A fixed combination hydrocodone IR dosage form could be prepared as follows. The process begins with the coated hydrocodone resinate as created in FIG. 4. The second drug component (pseudoephedrine HCl) is created as an Intermediate as shown in Example 6 and FIG. 6. In the last step, as shown in FIG. 10, the coated resinate, the Pseudoephedrine Intermediate component and the release enhancer pre-mix (prepared by a process shown in FIG. 7) and the other excipients are blended and compressed into a tablet.

### Example 9

### IR Hydrocodone Mono-Substance Component Intermediate for Filling Hard Gelatin Capsules

| | |
|---|---|
| Hydrocodone Resinate Intermediate* | 32 mg |
| Release enhancing agent | 15 mg |
| Magnesium Stearate | 2 mg |
| Colloidal Silicon Dioxide | 3 mg |
| IR component weight | 52 mg |

*Equivalent to Hydrocodone Bitartrate 5 mg

A mono-substance hydrocodone IR intermediate for capsules could be prepared as follows. The process begins with the coated hydrocodone resinate as created in Example 3 and FIG. 4. Next, the hydrocodone resinate intermediate, the release enhancing agent and the other excipients are blended to create an intermediate for filling capsules.

### Example 10

### ER Hydrocodone Mono-Substance Component Intermediate for Filling Hard Gelatin Capsules

| | |
|---|---|
| IR Coated Resin Intermediate* | 52 mg |
| Methacrylic Acid Copolymer | 20 mg |
| Talc | 2 mg |
| Triethyl Citrate | 3 mg |
| Colloidal Silicon Dioxide | 3 mg |
| Magnesium Stearate | 2 mg |
| ER component weight* | 82 mg |

*Equivalent to Hydrocodone Bitartrate 5 mg

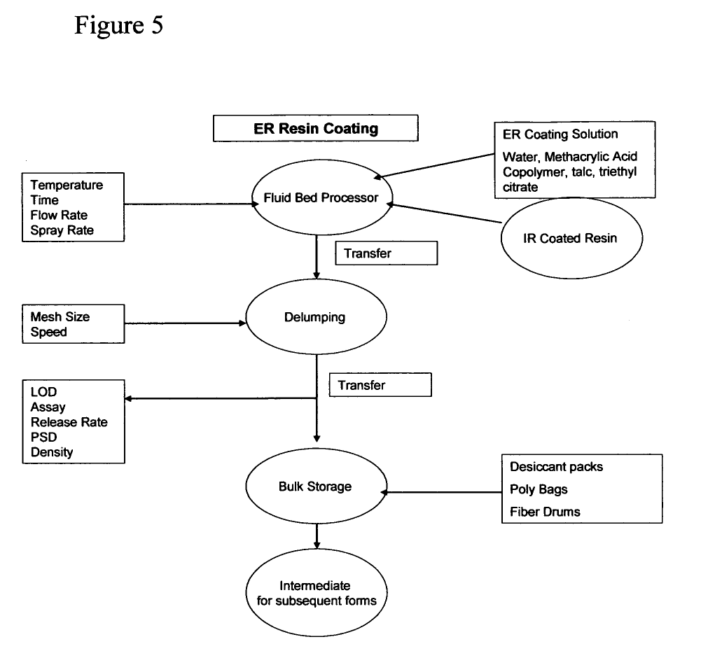

A mono-substance hydrocodone ER intermediate could be prepared as follows. The process begins with the coated hydrocodone resinate as created in Example 3 and FIG. 4. Next, the immediate release coated resinate intermediate is further coated as shown in FIG. 5 to create extended release (ER) intermediate particles.

### Example 11

### Formulation for a Commercial Product: IR/ER Hydrocodone Encapsulation to Fill a Hard Gelatin Capsule to Deliver 10 Mg Hydrocodone Total

| | |
|---|---|
| IR component weight* | 52 mg |
| ER component weight* | 82 mg |
| Empty Capsule Shell #1 | 76 mg |
| Total Dosage Form Weight | 210 mg* |

*Equivalent to Hydrocodone Bitartrate 5 mg

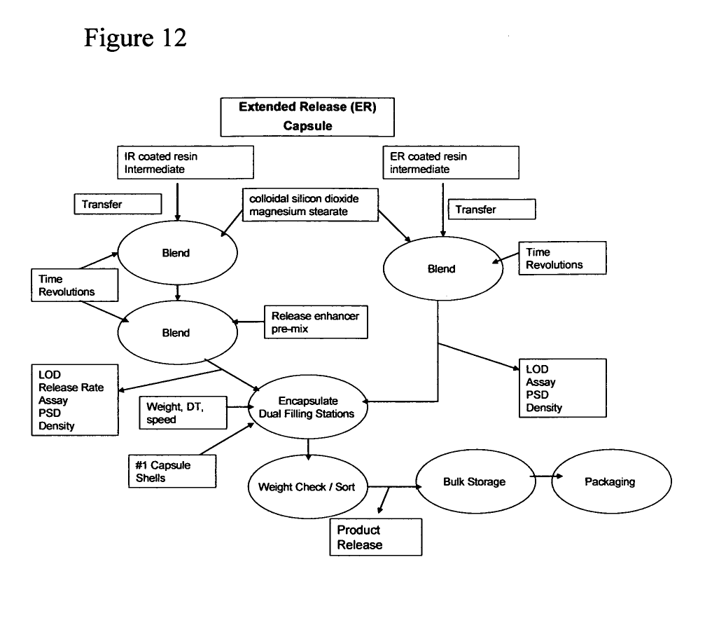

A mono-substance hydrocodone ER dosage form could be prepared as follows. The process for filling a capsule to create an extended release capsule (ER) is shown in FIG. 12. Notice that the process uses both the IR and ER coated hydrocodone intermediates from Examples 3 and 10, respectively.

### Example 12

### Mono-substance Codeine IR Intermediate

| | |
|---|---|
| Codeine Resinate Intermediate* | 60 mg |
| HPMC 6 cps | 3 mg |
| Talc | 2 mg |
| Triethyl Citrate | 2 mg |
| Total Dosage Form Weight | 67 mg |

*Equivalent to Codeine Phosphate 15 mg.

A mono-substance codeine IR intermediate could be prepared as follows. The process begins with the resination of the active ingredient. The process for creating the drug resinate is illustrated in FIG. 3. Next, the codeine resinate is coated to improve product flow as shown in FIG. 4.

### Example 13

### Formulation for a Commercial Product: IR Codeine Capsules

| | |
|---|---|
| Codeine Resinate Intermediate* | 67 mg |
| Release enhancing agent | 15 mg |
| Magnesium Stearate | 2 mg |
| Colloidal Silicon Dioxide | 3 mg |
| Empty Capsule Shell #3 | 48 mg |
| Total Dosage Form Weight | 135 mg |

*Equivalent to Codeine Phosphate 15 mg.

A mono-substance codeine IR dosage form could be prepared as follows. The process begins with the coated codeine resinate as created in Example 12 and FIG. 4. In the last step, as shown in FIG. 8, the coated codeine resinate, the release enhancing agent and the other excipients are blended and filled into hard gelatin capsules.

### Example 14

### Formulation for a Commercial Product: IR Codeine Tablets

| | |
|---|---|
| Codeine Resinate Intermediate* | 67 mg |
| Microcrystalline cellulose | 150 mg |
| Polyplasdone XL | 10 mg |
| Release enhancing agent | 15 mg |
| HPMC 6 cps | 20 mg |
| Anhydrous lactose | 100 mg |
| Magnesium stearate | 4 mg |
| Total Dosage Form Weight | 366 mg |

*Equivalent to Codeine Phosphate 15 mg

A mono-substance codeine IR dosage form could be prepared as follows. The process begins with the coated codeine resinate as created in Example 12 and FIG. 4. In the last step, as shown in FIG. 9, the coated codeine resinate, the release enhancing agent and the other excipients are blended and compressed into a tablet.

### Example 15

### Formulation for a Commercial Product: Fixed Combination Dosage Form Using the IR Codeine Component with a Second Medicinal Agent, Hard Gelatin Capsule

| | |
|---|---|
| Codeine Resinate Intermediate* | 67 mg |
| Pseudoephedrine HCl Intermediate | 358 mg |
| Microcrystalline Cellulose | 100 mg |
| Polyvinylpyrrolidone | 20 mg |
| Release enhancing agent | 15 mg |
| Magnesium Stearate | 4 mg |
| Colloidal Silicon Dioxide | 4 mg |
| Empty Capsule Shell #1 | 76 mg |
| Total Dosage Form Weight | 644 mg |

*Equivalent to Codeine Phosphate 15 mg

A fixed combination codeine IR dosage form could be prepared as follows. The process begins with the coated codeine resinate as created in Example 12 and FIG. 4. The second drug component (pseudoephedrine HCl) is created as an intermediate as shown in Example 6 and FIG. 6. In the last step, as shown in FIG. 11, the coated codeine resinate, the pseudoephedrine component intermediate and the release enhancing agent and the other excipients are blended and filled into hard gelatin capsules.

### Example 16

### Formulation for a commercial product: Fixed combination dosage form using the IR Codeine Component with a Second Medicinal Agent, Compressed Tablet

| | |
|---|---|
| Codeine Resinate Intermediate* | 67 mg |
| Pseudoephedrine HCl Intermediate** | 358 mg |
| Microcrystalline Cellulose | 250 mg |
| Polyvinylpyrrolidone | 20 mg |
| Release enhancing agent | 15 mg |
| Magnesium Stearate | 4 mg |
| Colloidal Silicon Dioxide | 4 mg |
| HPMC 6 cps | 20 mg |
| Total Dosage Form Weight | 738 mg |

Equivalent to Codeine Phosphate 15 mg
**Equivalent to Pseudoephedrine HCl 60 mg

A fixed combination codeine IR dosage form could be prepared as follows. The process begins with the coated codeine resinate as created in Example 12 and FIG. 4. The second drug component (pseudoephedrine HCl) is created as an Intermediate as shown in Example 6 and FIG. 6. In the last step, as shown in FIG. 10, the coated resinate, the second component Intermediate and the release enhancing agent and the other excipients are blended and compressed into a tablet.

### Example 17

### IR Codeine Mono-Substance Component Intermediate for Filling Hard Gelatin Capsules

| | |
|---|---|
| Codeine Resinate Intermediate* | 67 mg |
| Release enhancing agent | 15 mg |
| Magnesium Stearate | 2 mg |
| Colloidal Silicon Dioxide | 3 mg |
| IR component weight | 87 mg |

*Equivalent to Codeine Phosphate 15 mg

A mono-substance codeine IR intermediate could be prepared as follows. The process begins with the coated codeine resinate as created in Example 12 and FIG. 4. Next, the additional excipients are blended with the coated codeine resinate to produce the intermediate.

### Example 18

### ER Codeine Mono-Substance Component Intermediate for Filling Hard Gelatin Capsules

| | |
|---|---|
| IR Codeine Resin component weight* | 87 mg |
| Methacrylic Acid Copolymer | 20 mg |
| Talc | 2 mg |
| Triethyl Citrate | 3 mg |
| Colloidal Silicon Dioxide | 3 mg |
| Magnesium Stearate | 2 mg |
| ER component weight | 117 mg |

*Equivalent to Codeine Phosphate 15 mg

A mono-substance codeine ER intermediate could be prepared as follows. The process begins with the coated codeine resinate as created in Example 12 and FIG. 4. Next, the immediate release coated codeine resinate intermediate is further coated as shown in FIG. 5 to create an extended release (ER) intermediate.

### Example 19

### Formulation for a Commercial Product: IR/ER Codeine Encapsulation to Fill a Hard Gelatin Capsule to Deliver 30 Mg

| | |
|---|---|
| IR component weight* | 87 mg |
| ER component weight* | 117 mg |
| Empty Capsule Shell #1 | 76 mg |
| Total Dosage Form Weight | 280 mg* |

*Equivalent to Codeine Phosphate 10 mg

A mono-substance codeine ER dosage form could be prepared as follows. The process for filling a capsule to create an extended release capsule (ER) is shown in FIG. 12. Notice that the process uses both the IR and ER coated intermediates from Examples 17 and 18, respectively.

### Example 20

### ER Pseudoephedrine Component Intermediate for Filling Hard Gelatin Capsules

| | |
|---|---|
| IR Pseudoephedrine HCl* | 358 mg |
| Methacrylic Acid Copolymer | 20 mg |
| Talc | 2 mg |
| Triethyl Citrate | 3 mg |
| Colloidal Silicon Dioxide | 3 mg |
| Magnesium Stearate | 2 mg |
| ER component weight | 388 mg |

*Equivalent to Pseudoephedrine HCl 60 mg

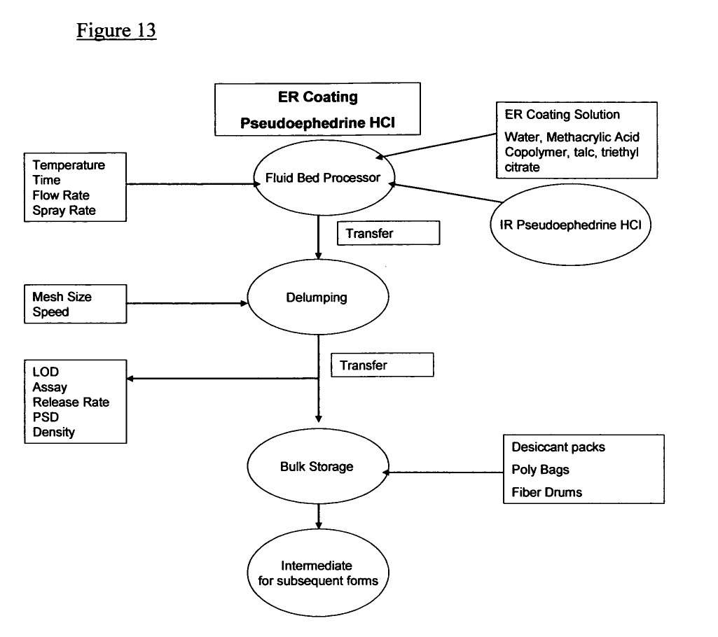

A mono-substance pseudoephedrine ER intermediate could be prepared as follows. The process for creating the ER pseudoephedrine intermediate is illustrated in FIG. 13. The composition of thee pseudoephedrine IR component is taken from Example 6 and FIG. 6.

### Example 21

### Formulation for a Commercial Product: IR/ER Hydrocodone, IR/ER Pseudoephedrine encapsulation to fill a hard gelatin capsule to deliver hydrocodone 10 mg, Pseudoephedrine 120 mg

| | |
|---|---|
| IR Hydrocodone component weight* | 52 mg |
| ER Hydrocodone component weight* | 82 mg |
| IR Pseudoephedrine component weight** | 358 mg |
| ER pseudoephedrine component weight** | 388 mg |
| Empty Capsule Shell #00 | 130 mg |
| Total Dosage Form Weight | 1,010 mg* |

*Equivalent to 5 mg hydrocodone
**Equivalent to 60 mg pseudoephedrine HCl

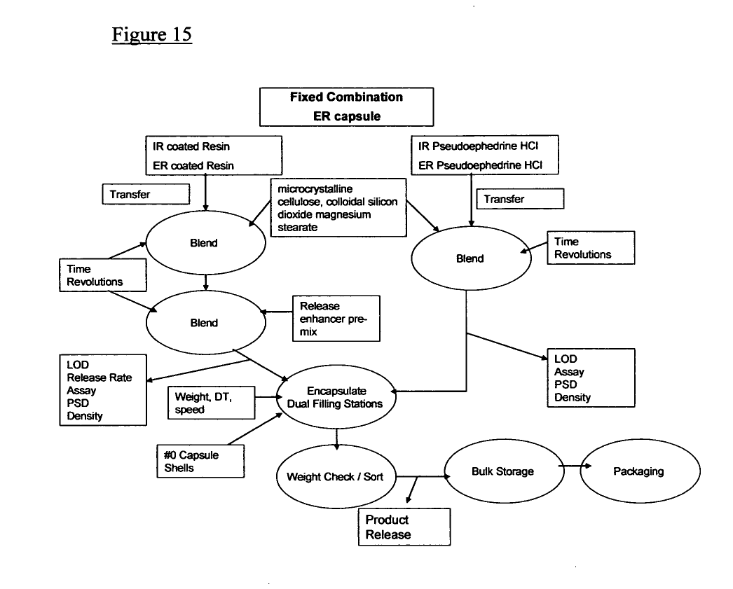

A fixed combination hydrocodone ER dosage form could be prepared as follows. The process for filling a capsule to create an extended release capsule (ER) fixed combination capsule is shown in FIG. 15. Notice that the process uses both the IR and ER coated hydrocodone intermediates from Examples 7 and 8, respectively and both the IR and ER pseudoephedrine intermediates from Examples 6 and 17, respectively.

### Example 22

### Formulation for a Commercial Product: IR/ER Hydrocodone, IR/ER Pseudoephedrine to Deliver Hydrocodone 10 Mg, Pseudoephedrine 120 Mg, Compressed Tablet

| | |
|---|---|
| IR Hydrocodone Resinate Intermediate* | 52 mg |
| ER Hydrocodone Resinate Intermediate* | 82 mg |
| IR Pseudoephedrine HCl Intermediate** | 358 mg |
| ER Pseudoephedrine HCl Intermediate** | 388 mg |
| Total Dosage Form Weight | 880 mg |

*Equivalent to 5 mg hydrocodone
**Equivalent to 60 mg pseudoephedrine HCl

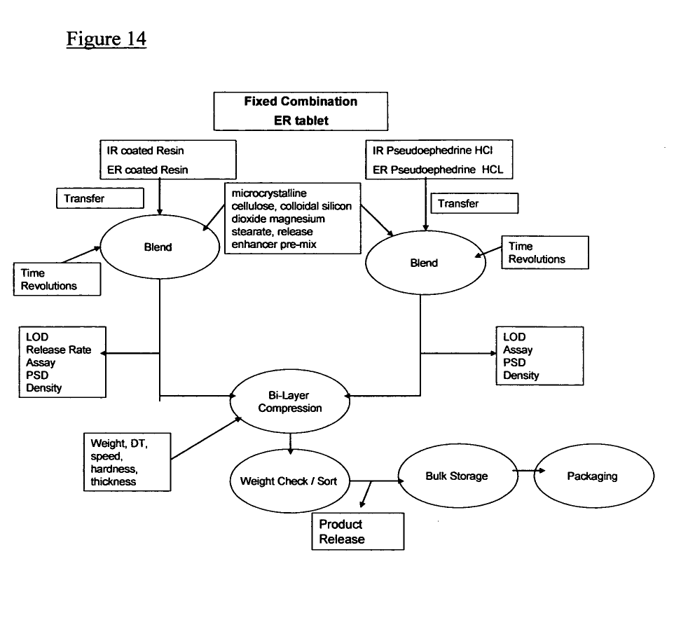

A fixed combination hydrocodone ER dosage form could be prepared as follows. The process for creating a compressed tablet to create an extended release (ER), fixed combination tablet is shown in FIG. 14. Notice that the process uses both the IR and ER coated hydrocodone intermediates for from Examples 7 and 8, respectively and both the IR and ER pseudoephedrine intermediates from Examples 6 and 17, respectively.

### Example 23

### Formulation for a Commercial Product: IR/ER Codeine, IR/ER Pseudoephedrine encapsulation to fill a hard gelatin capsule to deliver codeine 30 mg, Pseudoephedrine 120 Mg

| | |
|---|---|
| IR Codeine Resinate Intermediate* | 87 mg |
| ER Codeine Resinate Intermediate* | 117 mg |
| IR Pseudoephedrine Intermediate** | 358 mg |
| ER pseudoephedrine Intermediate** | 388 mg |
| Empty Capsule Shell #00 | 130 mg |
| Total Dosage Form Weight | 1,080 mg |

*Equivalent to 15 mg codeine
**Equivalent to 60 mg pseudoephedrine HCl

A fixed combination codeine ER dosage form could be prepared as follows. The process for filling a capsule to create an extended release capsule (ER) fixed combination capsule is shown in FIG. 15. Notice that the process uses both the IR and ER coated intermediates for codeine as shown in Examples 17 and 18, respectively and both the IR and ER pseudoephedrine intermediates from Examples 6 and 17, respectively.

### Example 24

### Formulation for a Commercial Product: IR/ER Codeine, ER/ER Pseudoephedrine to Deliver Codeine 30 Mg, Pseudoephedrine 120 Mg, Compressed Tablet

| | |
|---|---|
| IR Codeine Coated Resinate* | 87 mg |
| ER Codeine Coated Resinate* | 117 g |
| IR Pseudoephedrine HCl** | 358 mg |
| ER Pseudoephedrine HCl component** | 388 mg |
| Total Dosage Form Weight | 950 mg |

*Equivalent to 15 mg codeine
**Equivalent to 60 mg pseudoephedrine HCl

A fixed combination codeine ER dosage form could be prepared as follows. The process for creating a compressed tablet to create an extended release (ER), fixed combination tablet is shown in FIG. 14. Notice that the process uses both the IR and ER coated intermediates for codeine as shown in Examples 17 and 18, respectively and both the IR and ER pseudoephedrine intermediates from Examples 6 and 17, respectively.

### Other Embodiments

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmaceutics, pharmacology, endocrinology, or related fields are intended to be within the scope of the invention.

What is claimed is:

1. A solid oral dosage pharmaceutical composition comprising (i) at least one pharmaceutically active agent bound to a first strong acid ion exchange resin to form a strong acid ion exchange resinate and (ii) a release-enhancing agent; wherein said composition is capable of immediate release of said at least one pharmaceutically active agent from said strong acid ion exchange resinate.

2. The solid oral dosage pharmaceutical composition of claim 1, wherein said strong acid ion exchange resin is selected from the group consisting of a resin terminated in a sulphonic acid moiety, Amberlite IRP69, DOWEX 88, and DOWEX 50WX8.

3. The solid oral dosage pharmaceutical composition of claim 1, wherein said release-enhancing agent is selected from the group consisting of an inorganic salt, an organic base, a cationic surfactant, and a non-ionic surfactant.

4. The solid oral dosage pharmaceutical composition of claim 1 useful for oral administration to a patient having a condition selected from the group consisting of Helicobacter pylori infection and atrophic gastritis

5. The solid oral dosage pharmaceutical composition of claim 1 useful for administration to a patient within 24 hours of administration to said patient of a compound selected from the group consisting of a proton pump inhibitor, an H2 receptor antagonist, and an antacid.

6. The solid oral dosage pharmaceutical composition of claim 1 useful for administration to a patient having a condition selected from the group consisting of hypochlorhydria and achlorhydria in the stomach.

7. The solid oral dosage pharmaceutical composition of claim 3, wherein said release-enhancing agent is an inorganic salt comprising an ion selected from the group consisting of Fe³⁺, Ca²⁺, Mg²⁺, and Fe²⁺.

8. The solid oral dosage pharmaceutical composition of claim 3, wherein said release-enhancing agent is an organic base selected from the group consisting of thiamine, guanine, and cytosine.

9. The solid oral dosage pharmaceutical composition of claim 3, wherein said release-enhancing agent is a cationic surfactant selected from the group consisting of cetyltrimethylammonium bromide (CTAB), denatonium benzoate, and benzalkonium chloride.

10. The solid oral dosage pharmaceutical composition of claim 3, wherein said release-enhancing agent is a non-ionic surfactant selected from the group consisting of Tween 20 and Tween 80.

11. The solid oral dosage pharmaceutical composition of claim 1, wherein said composition is formulated as a capsule, a powder, a thin film, a caplet or a tablet.

12. The solid oral dosage pharmaceutical composition of claim 11, wherein said composition is formulated as a capsule.

13. The solid oral dosage pharmaceutical composition of claim 11, wherein said composition is formulated as a tablet.

14. The solid oral dosage pharmaceutical composition of claim 13, comprising about 1 mg to 10 mg of hydrocodone and about 5 mg to 100 mg of CaCl₂.

15. The solid oral dosage pharmaceutical composition of claim 14, comprising about 30 mg to 60 mg pseudoephedrine hydrochloride.

16. The solid oral dosage pharmaceutical composition of claim 13, comprising an immediate release component and an extended release component, said immediate release component including about 1 mg to 10 mg hydrocodone bound to said first strong acid ion exchange resin, about 5 mg to 100 mg of CaCl₂ and about 30 mg to 60 mg pseudoephedrine hydrochloride, said extended release component including about 1 mg to 10 mg hydrocodone and about 80 mg to 120 mg of pseudoephedrine hydrochloride.

17. The solid oral dosage pharmaceutical composition of claim 12, comprising about 1 mg to 10 mg hydrocodone and about 5 mg to 100 mg of CaCl₂.

18. The solid oral dosage pharmaceutical composition of claim 12, comprising about 1 mg to 10 mg hydrocodone bound to said first strong acid ion exchange resin, about 5 mg to 100 mg mg of CaCl₂ and about 30 mg to 60 mg pseudoephedrine hydrochloride.

19. The solid oral dosage pharmaceutical composition of claim 1, wherein at least two pharmaceutically active agents are bound to said first strong acid ion exchange resin.

20. The solid oral dosage pharmaceutical composition of claim 1, further comprising a second ion exchange resin; wherein said second ion exchange resin is bound to a pharmaceutically active agent and is coated with an extended release coating.

21. The solid oral dosage pharmaceutical composition of claim 20, wherein said second ion exchange resin is bound to the same pharmaceutically active agent as the first strong acid ion exchange resin.

22. The solid oral dosage pharmaceutical composition of claim 1, comprising an immediate release component and an extended release component, said immediate release component including about 1 mg to 10 mg hydrocodone bound to said first strong acid ion exchange resin, about 5 mg to 100 mg of CaCl₂ and about 30 mg to 60 mg pseudoephedrine hydrochloride, said extended release component including about 1 mg to 10 mg hydrocodone, and about 80 mg to 120 mg of pseudoephedrine hydrochloride.

23. The solid oral dosage pharmaceutical composition of claim 1, comprising an immediate release component and an extended release component, said immediate release component including about 5 mg to 15 mg codeine bound to said first strong acid ion exchange resin, about 15 mg to 120 mg of CaCl₂, and about 30 mg to 60 mg pseudoephedrine hydrochloride, said extended release component comprising about 5 mg to 15 mg codeine and about 80 mg to 120 mg of pseudoephedrine.

24. The solid oral dosage pharmaceutical composition of claim 1, comprising about 5 mg to 15 mg codeine bound to said first strong acid ion exchange resin and about 15 mg to 120 mg of CaCl₂; and wherein said composition is formulated as a capsule.

25. The solid oral dosage pharmaceutical composition of claim 1, comprising about 5 mg to 15 mg codeine bound to said first strong acid ion exchange resin and about 15 mg to 120 mg of CaCl₂; and wherein said composition is formulated as a tablet.

26. The solid oral dosage pharmaceutical composition of claim 1, comprising about 5 mg to 15 mg codeine bound to said first strong acid ion exchange resin, about 15 mg to 120 mg of CaCl₂ and about 30 mg to 60 mg pseudoephedrine hydrochloride; wherein said composition is formulated as a capsule.

27. The solid oral dosage pharmaceutical composition of claim 1, said composition further comprising about 5 mg to 15 mg codeine bound to said first strong acid ion exchange resin, about 15 mg to 120 mg of CaCl₂ and about 30 mg to 60 mg pseudoephedrine hydrochloride; wherein said composition is formulated as a tablet.

28. A method of treating a patient for a condition with a pharmaceutically active agent effective for treating said condition, said method comprising orally administering the composition of claim 1.

29. A method of treating a patient having a first condition and a second condition with a pharmaceutically active agent effective for treating said second condition, said method comprising the step of administering the solid oral dosage pharmaceutical composition of claim 1, wherein said first condition is selected from the group consisting of Helicobacter pylori infection, atrophic gastritis, hypochlorhydria and achlorhydria in the stomach; and wherein said second condition is a condition other than said first condition.

30. The method of claim 29, wherein the patient has within the past 24 hours been administered a compound selected from the group consisting of a proton pump inhibitor, an H2 receptor antagonist, and an antacid.

31. A method of delivering a pharmaceutically active agent to a patient, said method comprising orally administering the composition of claim 1 and a compound selected from the group consisting of a proton pump inhibitor, an H2 receptor antagonist, and an antacid, wherein said administration results in immediate release of said pharmaceutically active agent from said strong acid ion exchange resinate; and wherein said composition of claim 1 is administered within 24 hours of said proton pump inhibitor, H2 receptor antagonist, or antacid.
